(19) United States
(12) Patent Application Publication
(10) Pub. No.: US 2025/0353822 A1
(43) Pub. Date: Nov. 20, 2025

(54) METHOD FOR PRODUCING CUMENE, APPARATUS FOR PRODUCING CUMENE, AND METHOD FOR PRODUCING PROPYLENE OXIDE

(71) Applicant: Sumitomo Chemical Company, Limited, Chuo-ku, Tokyo (JP)

(72) Inventor: Shinjiro ISHIHARA, Ichihara-Shi, Chiba (JP)

(21) Appl. No.: 18/877,595

(22) PCT Filed: Jul. 4, 2023
(86) PCT No.: PCT/JP2023/024726

§ 371 Date: Jul. 14, 2025

(30) Foreign Application Priority Data
Jul. 7, 2022 (JP) 2022-109786

(51) Int. Cl. C07D 301/12; C07C 1/20; C07C 7/04

(52) U.S. Cl. CPC C07D 301/12; C07C 1/20; C07C 7/04

(57) ABSTRACT

A method for producing cumene of the present invention includes steps (e) and (f) below, a cumene conversion step (e): a step of converting cumyl alcohol to cumene to obtain a solution (1) containing cumene, the solution (1) being taken as a flow (1), and a cumene purification step (f): a step of separating the solution (1) of the flow (1) into at least a solution (2) containing purified cumene, and a solution (3) containing 2,3-dimethyl-2,3-diphenylbutane to obtain a flow (2) containing purified cumene and a flow (3) containing 2,3-dimethyl-2,3-diphenylbutane, in which the flow (3) contains 20 wt % or more and 99 wt % or less of acetophenone, and 1 wt % or more and 10 wt % or less of 2,3-dimethyl-2,3-diphenylbutane.

### CROSS-REFERENCE TO RELATED APPLICATION

This application claims priority to Japanese Patent Application No. 2022-109786, the disclosure of which is incorporated herein by reference in its entirety.

### FIELD

The present invention relates to a method for producing cumene, an apparatus for producing cumene, and a method for producing propylene oxide.

### BACKGROUND

Conventionally, a method for producing propylene oxide through an oxidation step of oxidizing cumene to obtain cumene hydroperoxide, an epoxidation step of reacting propylene with cumene hydroperoxide obtained in the oxidation step to obtain propylene oxide and cumyl alcohol, and a cumene conversion step of converting the cumyl alcohol obtained in the epoxidation step to cumene is known. Further, in the production method, it is known that cumene obtained in the cumene conversion step may be recycled to the oxidation step.

In the cumene conversion step, which converts cumyl alcohol to cumene, cumene is partly dimerized so that cumene dimer, such as 2,3-dimethyl-2,3-diphenylbutane is secondarily produced. However, in a flow containing cumene dimer, during transporting to the subsequent process, problems such as clogging of piping due to solidification of the components including cumene dimer occurred.

To solve the above problem, for example, Patent Literature 1 discloses a method of adding and mixing a diluent oil with a flow containing cumene dimer to hold the flow at the temperature of 50° C.

### CITATION LIST

### Patent Literature

Patent Literature 1: JP 2003-40810 A

### SUMMARY

### Technical Problem

However, in the method of Patent Literature 1, although the flow containing cumene dimer (e.g., 2,3-dimethyl-2,3-diphenylbutane) maintains good flowability at a temperature of 50° C., the flowability decreases at normal temperature. In an actual plant, although the temperature of the flow immediately after discharge from a distillation column is about 150° C., it is cooled thereafter to normal temperature in the piping to a mixing facility, a waste oil combustion facility, or the like. Therefore, in the method of Patent Literature 1, it was necessary to heat the piping or the like to the subsequent facility.

The present invention has been made in view of such circumstances, and it is an object of the present invention to provide a method for producing cumene, an apparatus for producing cumene, and a method for producing propylene oxide that allow a flow including 2,3-dimethyl-2,3-diphenylbutane to be capable of maintaining good flowability even at normal temperature.

### Solution to Problem

A method for producing cumene including steps (e) and (f) below,
a cumene conversion step (e): a step of converting cumyl alcohol to cumene to obtain a solution (1) containing cumene, the solution (1) being taken as a flow (1), and
a cumene purification step (f): a step of separating the solution (1) of the flow (1) into at least a solution (2) containing purified cumene, and a solution (3) containing 2,3-dimethyl-2,3-diphenylbutane to obtain a flow (2) containing purified cumene and a flow (3) containing 2,3-dimethyl-2,3-diphenylbutane, in which
the flow (3) contains 20 wt % or more and 99 wt % or less of acetophenone, and 1 wt % or more and 10 wt % or less of 2,3-dimethyl-2,3-diphenylbutane.

An apparatus for producing cumene according to the present invention is an apparatus for producing cumene using the method for producing cumene described above, the apparatus including:
a facility A for separating the solution (1) of the flow (1) into at least a solution (2) containing purified cumene and a solution (3) containing 2,3-dimethyl-2,3-diphenylbutane.

A method for producing propylene oxide according to the present invention is a method for producing propylene oxide including the aforementioned method for producing cumene, the method for producing propylene oxide including steps (a) to (f) below,
an oxidation step (a): a step of oxidizing cumene to obtain cumene hydroperoxide,
an epoxidation step (b): a step of reacting propylene with cumene hydroperoxide obtained in the oxidation step (a) to obtain a reaction mixture containing propylene oxide and cumyl alcohol,
a separation step (c): a step of separating a mixture containing propylene oxide from the reaction mixture containing propylene oxide and cumyl alcohol obtained in the epoxidation step (b) to obtain a residue containing cumyl alcohol,
a propylene oxide purification step (d): a step of distilling the mixture containing propylene oxide separated by the separation step (c) to obtain purified propylene oxide,
a cumene conversion step (e): a step of converting cumyl alcohol in the residue containing cumyl alcohol obtained in the separation step (c) to cumene to obtain a solution (1) containing cumene, the solution (1) being taken as a flow (1), and
a cumene purification step (f): a step of separating the solution (1) of the flow (1) into at least a solution (2) containing purified cumene, and a solution (3) containing 2,3-dimethyl-2,3-diphenylbutane to obtain a flow (2) containing purified cumene and a flow (3) containing 2,3-dimethyl-2,3-diphenylbutane.

### Effect of the Invention

According to the present invention, it is possible to provide a method for producing cumene, an apparatus of producing cumene, and a method for producing propylene oxide that allow a flow containing 2,3-dimethyl-2,3-diphenylbutane to be capable of maintaining good flowability even at normal temperature.

### BRIEF DESCRIPTION OF DRAWINGS

FIG. 1A to FIG. 1C are explanatory diagrams for explaining examples of a facility A included in a cumene production apparatus according to a present embodiment.

FIG. 2A to FIG. 2C are explanatory diagrams for explaining examples of a facility D included in a cumene production apparatus according to the present embodiment.

### DESCRIPTION OF EMBODIMENTS

Although an embodiment of the present invention will be hereinafter described, the present invention is not limited to the following embodiment.

### <Method for Producing Cumene>

A method for producing cumene according to this embodiment includes steps (e) and (f) below,
a cumene conversion step (e): a step of converting cumyl alcohol to cumene to obtain a solution (1) containing cumene, the solution (1) being taken as a flow (1), and
a cumene purification step (f): a step of separating the solution (1) of the flow (1) into at least a solution (2) containing purified cumene, and a solution (3) containing 2,3-dimethyl-2,3-diphenylbutane to obtain a flow (2) containing purified cumene and a flow (3) containing 2,3-dimethyl-2,3-diphenylbutane.

Examples of the cumene conversion step (e) include: a step of obtaining cumene by dehydrating cumyl alcohol and then reacting it with hydrogen, in the presence of a catalyst; and a step of obtaining cumene by reacting cumyl alcohol with hydrogen to cause hydrocracking in the presence of a catalyst, and the like. Cumyl alcohol means 2-phenyl-2-propanol.

The cumene conversion step (e) is preferably carried out in the presence of carbon monoxide. The concentration of carbon monoxide is preferably 0.1 to 10 volume %, more preferably 0.5 to 5 volume %.

In one mode, the cumene conversion step includes a step of dehydrating cumyl alcohol to obtain a mixture containing α-methylstyrene in the presence of a catalyst (hereinafter referred to as “dehydration step”), and a step of contacting hydrogen with the mixture containing α-methylstyrene obtained in the dehydration step and thereby allowing α-methylstyrene in the mixture to react with hydrogen to obtain a conversion mixture containing cumene (hereinafter referred to as “hydrogenation step”).

In yet another mode, the cumene conversion step is a step of contacting hydrogen with a residue containing cumyl alcohol in the presence of a catalyst and thereby allowing cumyl alcohol in the residue to react with hydrogen to obtain a conversion mixture containing cumene (hereinafter referred to as “hydrogenolysis step”).

First, the description will be hereinafter given for a mode in which the cumene conversion step includes the dehydration step and the hydrogenation step.

Examples of the catalyst used in the dehydration step (hereinafter referred to as “dehydration catalyst”) include a homogeneous acid catalyst such as sulfuric acid, phosphoric acid, or p-toluene sulfonic acid; and a solid acid catalyst such as active alumina, titania, zirconia, silica alumina, zeolite, or the like. The dehydration catalyst is preferably a solid acid catalyst, more preferably an active alumina from the viewpoint of improving reaction efficiency.

The dehydration reaction in the dehydration step is usually carried out by contacting cumyl alcohol with the dehydration catalyst. In one mode, cumyl alcohol may be contacted with the dehydration catalyst in the presence of hydrogen for hydrogenation reaction in the hydrogenation step subsequent to the dehydration reaction. The dehydration reaction can be carried out in a liquid phase in the presence of a solvent. The solvent must be substantially inert to a reaction raw material and a product thereof. The solvent may be a substance present in the residue containing cumyl alcohol to be used. For example, when the residue containing cumyl alcohol contains cumene, this cumene can be served as a solvent and other solvents need not to be used. Usually, the dehydration reaction temperature is preferably 50 to 450° C., more preferably 150 to 300° C. Usually, the dehydration reaction pressure is preferably 10 to 10000 kPa-G, more preferably 500 to 4000 kPa-G, even more preferably 1000 to 2000 kPa-G.

Examples of the catalyst used in the hydrogenation step (hereinafter referred to as “hydrogenation catalyst”) include a catalyst containing a metal of Group 10 or Group 11 in the periodic table, and more specific examples thereof include a catalyst containing nickel, a catalyst containing palladium, a catalyst containing platinum, and a catalyst containing copper. The hydrogenation catalyst is preferably a catalyst containing nickel, a catalyst containing palladium, or a catalyst containing copper from the viewpoint of inhibiting nucleus hydrogenation reaction of an aromatic ring and realizing high yield. The catalyst containing nickel is preferably nickel, nickel alumina, nickel silica, or nickel carbon. The catalyst containing palladium is preferably palladium alumina, palladium silica, or palladium carbon. The catalyst containing copper is preferably copper, Raney copper, copper chrome, copper zinc, copper chrome zinc, copper silica, or copper alumina. These catalysts may be used alone or in combination.

The hydrogenation reaction in the hydrogenation step is carried out by contacting the hydrogenation catalyst with α-methylstyrene and hydrogen. In one mode, the hydrogenation reaction is carried out subsequent to the dehydration reaction, in which a part of water generated in the dehydration reaction may be separated by oil water separation or the like, or a part of water is not separated to be allowed to contact with the hydrogenation catalyst along with α-methylstyrene. The amount of hydrogen required for the hydrogenation reaction may be equimolar with α-methylstyrene, but since the mixture containing α-methylstyrene obtained in the dehydration step usually contains a component other than α-methylstyrene that consumes hydrogen, excess hydrogen is used.

The higher the partial pressure of hydrogen, the faster the reaction proceeds. Thus, usually, the molar ratio of hydrogen/α-methylstyrene is preferably 1/1 to 20/1, more preferably 1/1 to 10/1, even more preferably 1/1 to 3/1. Usually, the molar ratio of hydrogen/(cumene+cumyl alcohol) is 1/25 or more. The molar ratio of hydrogen/(cumene+cumyl alcohol) may be more than 1/25. The excess hydrogen remaining after the hydrogenation reaction can also be recycled and used after separation from the reaction solution (conversion mixture). The amount of the substance “hydrogen” in the molar ratio is the amount of the substance of hydrogen to be subjected to the hydrogenation reaction, and the amount of the substances “cumene+cumyl alcohol” is the total amount of the substances of cumene and cumyl alcohol in the liquid to be subjected to the dehydration reaction.

A method for producing hydrogen used in the hydrogenation step is not particularly limited. For example, it is possible to use hydrogen made by the following production method. Hydrogen to be used is generally selected in consideration of price and environmental load.

Examples of the method for producing hydrogen include a method for steam reforming a fossil fuel such as natural gas or petroleum, aqueous shift reaction of carbon monoxide, electrolysis of water, electrolysis of salt, dehydrogenation of hydrocarbon, pyrolysis of methane, and producing as a by-product from soda electrolysis, producing as a by-product from iron works, and producing as a by-product from coal carbonization processes. Alternatively, a method for steam reforming of gas produced by decomposing biomass in the same manner as that for fossil fuels, a method for methane fermentation from biomass and further steam reforming of the methane, a method for directly producing hydrogen by fermentation of biomass, a method for decomposing water by photocatalyst, and the like are known. Examples of other methods include a method for decomposition of ammonia.

The hydrogenation reaction can be carried out in a liquid phase in the presence of a solvent or in a gas phase. The solvent must be substantially inert to a reaction raw material and a product thereof. The solvent may be a substance present in a mixture containing α-methylstyrene. For example, when the mixture containing α-methylstyrene contains cumene, cumene can be served as a solvent and other solvents need not to be used. Usually, the hydrogenation reaction temperature is preferably 0 to 500° C., more preferably 30 to 400° C., even more preferably 50 to 300° C. Usually, the hydrogenation reaction pressure is preferably 100 to 10000 kPa-G, more preferably 500 to 4000 kPa-G, even more preferably 1000 to 2000 kPa-G.

The dehydration and hydrogenation reactions can be advantageously carried out in the form of a slurry or fixed bed. In the case of large-scale industrial operation, it is preferable to use a fixed bed. The dehydration and hydrogenation reactions can also be carried out according to the reaction form such as by a batch method, a semi-continuous method, a continuous method, or the like. Separate reactors may be used respectively for the dehydration and hydrogenation reactions, or a single reactor may be used. The reactor in the continuous method is an adiabatic reactor or an isothermal reactor, but the adiabatic reactor is preferable because the equipment for heat removal is required in the isothermal reactor.

Next, a description will be given hereinafter for the mode where the cumene conversion step includes the hydrogenolysis step.

Examples of the catalyst used in the hydrogenolysis step (hereinafter referred to as “hydrogenolysis catalyst”) include a catalyst containing a metal of Group 9, Group 10, Group 11, or Group 12 in the periodic table, and more specific examples thereof include a catalysts containing cobalt, a catalyst containing nickel, a catalyst containing palladium, a catalyst containing copper, and a catalyst containing zinc. The hydrogenolysis catalyst is preferably a catalyst containing nickel, a catalyst containing palladium or a catalyst containing copper from the viewpoint of suppressing the formation of the by-product. Examples of the catalyst containing nickel include nickel, nickel alumina, nickel silica, and nickel carbon. Examples of the catalyst containing palladium include palladium-alumina, palladium-silica, palladium-carbon, and the like. Examples of the catalyst containing copper include copper, Raney copper, copper-chromium, copper-zinc, copper-chromium-zinc, copper-silica, copper-alumina, and the like. The hydrogenolysis reaction can be carried out in a liquid phase in the presence of a solvent or in a gas phase. The solvent must be substantially inert to a reaction raw material and a product thereof. The solvent may be a substance present in the residue containing cumyl alcohol to be used. For example, when the residue containing cumyl alcohol contains cumene, cumene can be served as a solvent and other solvents need not to be used. The amount of hydrogen required for the hydrogenolysis reaction may be equimolar with cumyl alcohol, but excess hydrogen is used because the residue containing cumyl alcohol obtained in the separation step (described later) contains a component other than cumyl alcohol that consumes hydrogen.

The higher the partial pressure of hydrogen, the faster the reaction proceeds. Thus, usually, the molar ration of hydrogen/cumyl alcohol is preferably 1/1 to 20/1, more preferably 1/1 to 10/1, even more preferably 1/1 to 3/1. Usually, the molar ratio of hydrogen/(cumene+cumyl alcohol) is 1/25 or more. The molar ratio of hydrogen/(cumene+cumyl alcohol) may be more than 1/25. The excess hydrogen remaining after the hydrogenolysis reaction can also be recycled and used after separation from the reaction liquid.

A method for producing hydrogen used in the hydrogenolysis step is not particularly limited. For example, it is possible to use hydrogen made by the following production method. Hydrogen to be used is generally selected in consideration of price and environmental load.

Examples of the method for producing hydrogen include a method for steam reforming a fossil fuel such as natural gas or petroleum, aqueous shift reaction of carbon monoxide, electrolysis of water, electrolysis of salt, dehydrogenation of hydrocarbon, pyrolysis of methane, and producing as a by-product from soda electrolysis, producing as a by-product from iron works, and producing as a by-product from coal carbonization processes. Alternatively, a method for steam reforming of gas produced by decomposing biomass in the same manner as that for fossil fuels, a method for methane fermentation from biomass and further steam reforming of the methane, a method for directly producing hydrogen by fermentation of biomass, a method for decomposing water by photocatalyst, and the like are known. Examples of other methods include a method for decomposition of ammonia.

Usually, the hydrogenolysis reaction temperature is preferably 0 to 500° C., more preferably 50 to 450° C., even more preferably 150 to 300° C. Usually, the hydrogenolysis reaction pressure is preferably 100 to 10000 kPa-G, more preferably 500 to 4000 kPa-G, even more preferably 1000 to 2000 kPa-G. The hydrogenolysis reaction can be advantageously carried out in the form of a slurry or fixed bed. In the case of large-scale industrial operation, it is preferable to use a fixed bed. Further, the hydrogenolysis reaction can be carried out according to the reaction form, such as by a batch method, a semi-continuous method, a continuous method, or the like.

The content of cumene in the conversion mixture containing cumene is usually preferably 90 wt % or more per 100 wt % of the conversion mixture containing cumene.

In the cumene purification step (f), a flow (3) contains 20 wt % or more and 99 wt % or less of acetophenone, and 1 wt % or more and 10 wt % or less of 2,3-dimethyl-2,3-diphenylbutane from the viewpoint of maintaining good flowability even at normal temperature. The normal temperature herein is 15 to 25° C.

The content of acetophenone contained in the flow (3) is preferably 25 wt % or more and 95 wt % or less, more preferably 30 wt % or more and 90 wt % or less from the viewpoint of maintaining good flowability even at normal temperature. The content of 2,3-dimethyl-2,3-diphenylbutane contained in the flow (3) is preferably 1.5 wt % or more and 9 wt % or less, more preferably 2 wt % or more and 8 wt % or less from the viewpoint of maintaining good flowability even at normal temperature.

Acetophenone formed in an oxidation step (a) (described later) or an epoxidation step (b) (described later) can be used as acetophenone contained in the flow (3). In this case, it is possible to increase the content of acetophenone contained in the flow (3) by, for example, increasing the reaction temperature in an oxidation step (a) (described later), decreasing the reaction temperature in an epoxidation step (b) (described later), increasing the concentration of carbon monoxide in the cumene conversion step (e), decreasing the temperature of a distillation column in the cumene purification step (f), or increasing the pressure of the distillation column. Further, it is possible to decrease the content of acetophenone contained in the flow (3) by, for example, decreasing the reaction temperature in the oxidation step (a) (described later), increasing the reaction temperature in the epoxidation step (b) (described later), decreasing the concentration of carbon monoxide in the cumene conversion step (e), increasing the temperature of a distillation column or decreasing the pressure of the distillation column in the cumene purification step (f). Acetophenone contained in the flow (3) may be one added to the flow (3).

2,3-dimethyl-2,3-diphenylbutane contained in the flow (3) can be formed by controlling the conditions in the cumene conversion step (e). For example, the content of 2,3-dimethyl-2,3-diphenylbutane contained in the flow (3) can be increased by decreasing the reaction temperature in the cumene conversion step (e). Further, for example, the content of 2,3-dimethyl-2,3-diphenylbutane contained in the flow (3) can be decreased by increasing the reaction temperature in the cumene conversion step (e).

The flow (3) may contain 0.1 wt % or more and 5 wt % or less of ethyl benzene from the viewpoint of better flowability. The content of ethylbenzene contained in the flow (3) is preferably 0.5 wt % or more and 4.5 wt % or less, more preferably 1.0 wt % or more and 4.0 wt % or less.

When the flow (3) contains ethylbenzene, one mode may be configured such that the cumene purification step (f) includes a step of separating a solution (1) of the flow (1) into a solution (2) containing purified cumene, a solution (3) containing 2,3-dimethyl-2,3-diphenylbutane, and a solution (3′) containing ethylbenzene, and a step of mixing at least a part of the solution (3) with at least a part of the solution (3′) to obtain a solution containing 2,3-dimethyl-2,3-diphenylbutane and ethylbenzene, the solution being taken as the flow (3). In the above mode, the separation of the solution (2), the solution (3) and the solution (3′) may be made in one stage or in two stages. The separation in two stages may be configured such that, for example, after the separation of the solution (3′), the solution (2) and the solution (3) are separated, or after the separation of the solution (3), the solution (2) and the solution (3′) are separated. Also, the above mode may be configured to include a step of recovering cumene from the solution (3) and/or the solution (3′) after the step of separating the flow into the solution (2), the solution (3), and solution (3′).

The flow (3) may further contain waste oil from the viewpoint of maintaining better flowability at normal temperature. As the waste oil, for example, it is possible to use waste oil obtained in a propylene oxide purification step (d) (described later). The flow (3) may further contain unreacted cumyl alcohol in the cumene conversion step (e).

### <Apparatus for Producing Cumene>

An apparatus for producing cumene according to this embodiment is an apparatus for producing cumene using the method for producing cumene described above. The cumene producing apparatus includes a facility A that separates the solution (1) of flow (1) into at least a solution (2) containing purified cumene and a solution (3) containing 2,3-dimethyl-2,3-diphenylbutane. The facility A may be configured to further separate the solution (1) of the flow (1) into the solution (3′) containing ethylbenzene. The facility A may include one distillation column or a plurality of distillation columns.

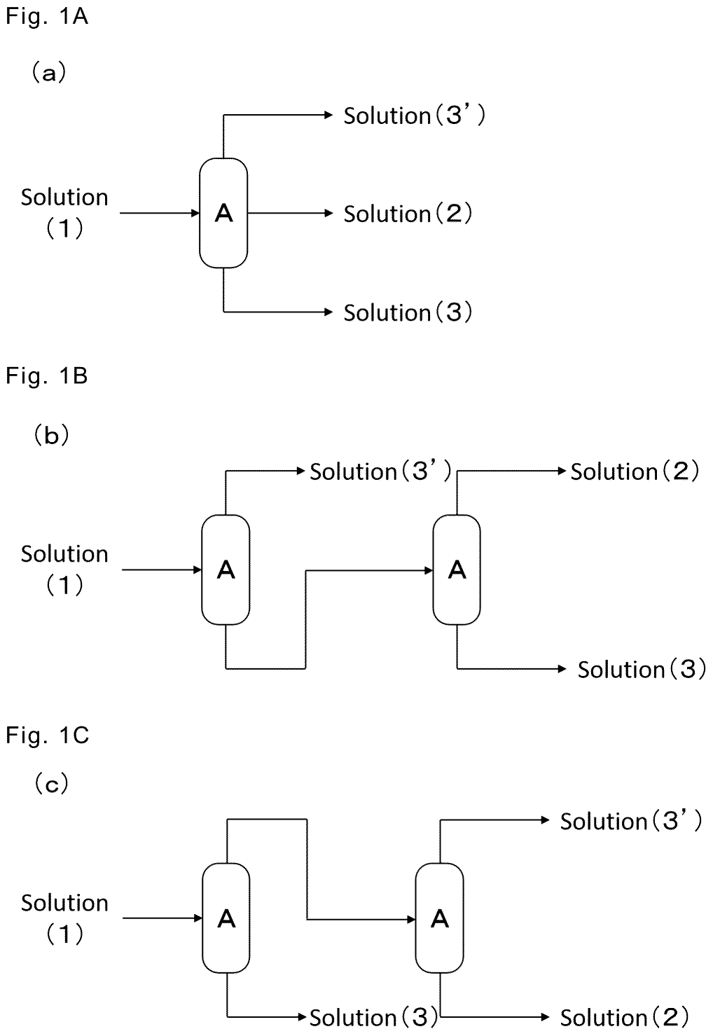

An example of the facility A will be described with reference to FIG. 1A to FIG. 1C. As shown in FIG. 1A, in one mode, the facility A includes one distillation column and configured to separate the solution (1) into the solution (2) containing purified cumene, the solution (3) containing 2,3-dimethyl-2,3-diphenylbutane, and the solution (3′) containing ethylbenzene in one stage. In this case, the solution (3′) containing ethylbenzene which is lightest is separated from an upper part of the facility A, and the solution (3) containing 2,3-dimethyl-2,3-diphenylbutane which is heaviest is separated from a lower part of the facility A.

As shown in FIG. 1B, in another mode, the facility A includes two distillation columns and configured to separate the solution (1) into the solution (2) containing purified cumene, the solution (3) containing 2,3-dimethyl-2,3-diphenylbutane, and the solution (3′) containing ethylbenzene in two stages. Specifically, after separating the solution (3′) from a lower part in the first distillation column, the solution (2) is separated from an upper part and the solution (3) is separated from a lower part, in the second distillation column.

As shown in FIG. 1C, in another mode, the facility A includes two distillation columns and configured to separate the solution (1) into the solution (2) containing purified cumene, the solution (3) containing 2,3-dimethyl-2,3-diphenylbutane, and the solution (3′) containing ethylbenzene in two stages. Specifically, after separating the solution (3) from a lower part in the first distillation column, the solution (3′) is separated from an upper part and the solution (2) is separated from a lower part, in the second distillation column.

The cumene producing apparatus according to this embodiment may further include a facility D for recovering cumene from the solution (3) and/or the solution (3′). The facility D is connected to the downstream side of the facility A. The facility D may include one distillation column or a plurality of distillation columns.

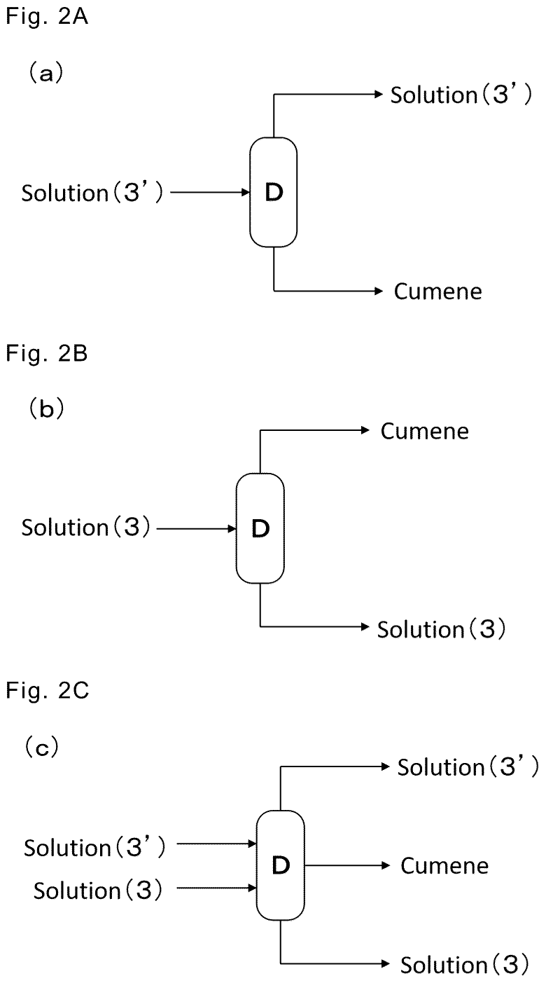

An example of the facility D will be described with reference to FIG. 2A to FIG. 2C. As shown in FIG. 2A, in one mode, the facility D includes one distillation column and configured to recover cumene from the solution (3′). The solution (3′) from which cumene has been recovered is separated from an upper part of the facility D.

As shown in FIG. 2B, in another mode, the facility D includes one distillation column and configured to recover cumene from the solution (3). The solution (3) from which cumene has been recovered is separated from a lower part of the facility D.

As shown in FIG. 2C, in still another mode, the facility D includes one distillation column and configured to recover cumene from the solution (3) and the solution (3′). The solution (3) from which cumene has been recovered is separated from a lower part of the facility D, and the solution (3′) from which cumene has been recovered is separated from an upper part of the facility D.

The cumene producing apparatus according to this embodiment may further include a facility B for mixing at least a part of the solution (3) with at least a part of the solution (3′), and a facility C connected to the facility B and configured to burn waste oil. In one mode of the cumene producing apparatus including the facilities B and C, the facility B is configured to mix at least a part of the solution (3′) with at least a part of the solution (3) separated in the facility A. Then, waste oil is burned in the facility C connected to the facility B to be waste oil′

### <Method for Producing Propylene Oxide>

A method for producing propylene oxide according to this embodiment is a method for producing propylene oxide including the method for producing cumene as described above, and including the steps of (a) to (f) below.
(a) Oxidation step: a step of oxidizing cumene to obtain cumene hydroperoxide.
(b) Epoxidation step: a step of reacting propylene with cumene hydroperoxide obtained in the oxidation step (a) to obtain a reaction mixture containing propylene oxide and cumyl alcohol.
(c) Separation step: a step of separating a mixture containing propylene oxide from the reaction mixture containing propylene oxide and cumyl alcohol obtained in the epoxidation step to obtain a residue containing cumyl alcohol.
(d) Propylene oxide purification step: a step of distilling the mixture containing propylene oxide separated by the separation step (c) to obtain purified propylene oxide.
(e) Cumene conversion step: a step of converting cumyl alcohol in the residue containing cumyl alcohol obtained in the separation step (c) to cumene to obtain a flow (1) containing cumene.
(f) Cumene purification step: a step of separating the flow (1) into at least a flow (2) containing purified cumene and a flow (3) containing 2,3-dimethyl-2,3-diphenylbutane.

In the oxidation step (a), cumene is oxidized to obtain cumene hydroperoxide. Oxidation of cumene is usually carried out by autoxidation with an oxygen-containing gas such as air, oxygen-enriched air, or the like. The oxidation reaction may be carried out without an additive or may be carried out with an additive such as alkali. The reaction temperature is usually 50 to 200° C. and the reaction pressure is usually between atmospheric pressure and 5 MPa.

Examples of the additive include an alkali metal hydroxide such as NaOH, KOH; an alkali earth metal hydroxide; an alkali metal carbonate such as Na₂CO₃, or NaHCO₃; ammonia; (NH₄)₂CO₃; an alkali metal ammonium carbonate, and the like.

In the epoxidation step (b), cumene hydroperoxide obtained in the oxidation step (a) is reacted with propylene to obtain a reaction mixture containing propylene oxide and cumyl alcohol. The epoxidation step (b) is preferably carried out in the presence of a catalyst containing a titanium-containing silicon oxide from the viewpoint of forming propylene oxide in high yield and high selectivity. The catalyst is preferably a so-called Ti-silica catalyst containing Ti chemically bonded to a silicon oxide. Examples of the Ti-silica catalyst include a catalyst prepared by supporting a Ti compound on a silica carrier, a catalyst prepared by combining a Ti compound with a silicon oxide by a co-precipitation method or sol gel method, a zeolite compound containing Ti, and the like.

The epoxidation reaction in the epoxidation step (b) is carried out by contacting propylene and cumene hydroperoxide with a catalyst. The molar ratio of propylene to cumene hydroperoxide (propylene/cumene hydroperoxide) is preferably 2/1 to 50/1. With the molar ratio being 2/1 or more, epoxidation can be carried out at a good reaction rate, and therefore the epoxidation reaction can be efficiently carried out. Further, with the molar ratio being 50/1 or less, the supply amount of propylene can be suppressed from being excessive, and therefore it is possible to suppress the energy required due to the step of recovering and recycling propylene.

The method for producing propylene used in the epoxidation step is not particularly limited, and it is possible to employ a method using propylene produced by the following production method.

Examples of the propylene production method include cracking of naphtha or ethane, fluid catalytic cracking of vacuum gas oil, dehydrogenation of propane, disproportionation of 2-butene with ethylene, MTO (Methanol to Olefin) reaction to convert methanol or dimethyl ether, Fisher-Tropsch (FT) synthesis method to react carbon monoxide with hydrogen, dehydration of isopropanol, and the like. Alternatively, it is possible to use propylene produced by a method of reducing the environmental burden, such as by a method of obtaining propylene from bioethanol made from plants and/or isopropanol, by a FT synthesis method using carbon dioxide and biomass, or by a method of catalytically decomposing waste plastics.

The epoxidation reaction can be carried out in a liquid phase using a solvent. The solvent should be liquid at the temperature and the pressure at the time of the reaction and substantially inert to the reactant and the product thereof. As the solvent, for example, cumene can be used. Examples of the solvent other than cumene include a monocyclic aromatic solvent (specifically, benzene, toluene, chlorobenzene, orthodichlorobenzene, etc.), an alkane (specifically, octane, decane, dodecane, etc.), and the like.

The epoxidation reaction temperature is usually 0 to 200° C. and is preferably 25 to 200° C. The pressure may be sufficient to keep the reaction mixture in a liquid state. It is advantageous that the pressure is usually 100 to 10000 kPa.

The epoxidation reaction can be advantageously carried out using a catalyst in the form of a slurry or fixed bed. In the case of large-scale industrial operations, it is preferable to use a fixed bed. The epoxidation reaction can also be carried out by a batch method, a semi-continuous method, or a continuous method.

The content of propylene oxide in the reaction mixture obtained in the epoxidation step (b) is preferably 1 to 31 wt %, more preferably 1 to 23 wt %. The content of cumyl alcohol in the reaction mixture is preferably 5 to 80 wt %, more preferably 5 to 60 wt %, even more preferably 5 to 40 wt %.

Cumyl alcohol formed in the epoxidation step (b) is supplied to the cumene conversion step (e) in the cumene production method described above. Usually, a solution containing cumyl alcohol after propylene oxide and unreacted propylene are recovered from the reaction mixture obtained in the epoxidation reaction is supplied into the cumene conversion step (e).

In the separation step (c), a mixture containing propylene oxide is separated from the reaction mixture containing propylene oxide and cumyl alcohol obtained in the epoxidation step (b) to obtain a residue containing cumyl alcohol. Examples of the separation method include a method using a distillation column (preferably a plurality of distillation columns). The pressure in the distillation column is preferably 100 to 5000 kPa, more preferably 100 to 3000 kPa. The column top temperature is preferably −50 to 150° C., more preferably 0 to 130° C. At this time, the column bottom temperature is preferably 50 to 230° C., more preferably 60 to 210° C.

The content of propylene oxide in the separated mixture containing propylene oxide is preferably 99 wt % or more per 100 wt % of the mixture. The content of cumyl alcohol in the residue containing cumyl alcohol is preferably 5 to 80 wt %, more preferably 5 to 60 wt %, even more preferably 5 to 40 wt % per 100 wt % of the residue.

The residue containing cumyl alcohol contains cumene, acetophenone, ethylbenzene, phenol, cymene or the like as a component other than cumyl alcohol.

In the propylene oxide purification step (d), the mixture containing propylene oxide separated by the separation step (c) is distilled to obtain purified propylene oxide. The mixture containing propylene oxide obtained by the separation step (c) usually contains water, hydrocarbon, and an oxygen-containing compound as impurities. Examples of the hydrocarbon include hydrocarbon with 3 to 7 carbon atoms. Examples of the oxygen-containing compound include methanol, acetaldehyde, acetone, propionaldehyde, methyl formate, and the like.

As a method for removing these impurities, known distillation techniques may be suitably combined, but from the viewpoint of efficiently removing water, hydrocarbon and an oxygen-containing compound, it is preferable to perform purification by combining extraction distillation in which hydrocarbon with 7 to 10 carbon atoms is served as an extractant with another distillation.

Examples of the hydrocarbon with 7 to 10 carbon atoms, which serves as the extractant, include a straight-chain saturated hydrocarbon such as n-heptane, n-octane, n-nonane, n-decane, or the like; and a branched saturated hydrocarbon such as 2,2-dimethylpentane, 2,3-dimethylpentane, 2,2-dimethylhexane, 2,3-dimethylhexane, or the like. These extractants can be used either alone or in a mixture of these compounds.

The type and operating conditions of the extraction distillation column and other distillation columns, the amount of extractant used, etc. can be determined as appropriate depending on the quality of the product required.

The cumene conversion step (e) and the cumene purification step (f) are the same as those in the method for producing cumene according to this embodiment. Cumyl alcohol in the cumene conversion step (e) is cumyl alcohol in the residue containing cumyl alcohol obtained in the separation step (c).

Cumene obtained in the cumene purification step (f) is recycled to the oxidation step (a). The obtained cumene may be recycled into the oxidation step (a) after purification by distillation, washing with water, etc.

The method for producing cumene, the apparatus for producing cumene, and the method for producing propylene oxide according to this embodiment are not limited to the above embodiment, and various changes can be made without departing from the scope of the present invention.

The present invention includes the following features.

[1] A method for producing cumene including steps (e) and (f) below,
a cumene conversion step (e): a step of converting cumyl alcohol to cumene to obtain a solution (1) containing cumene, the solution (1) being taken as a flow (1), and
a cumene purification step (f): a step of separating the solution (1) of the flow (1) into at least a solution (2) containing purified cumene, and a solution (3) containing 2,3-dimethyl-2,3-diphenylbutane to obtain a flow (2) containing purified cumene and a flow (3) containing 2,3-dimethyl-2,3-diphenylbutane, in which
the flow (3) contains 20 wt % or more and 99 wt % or less of acetophenone, and 1 wt % or more and 10 wt % or less of 2,3-dimethyl-2,3-diphenylbutane.
   [2] The method for producing cumene according to [1] above, wherein the flow (3) further contains 0.1 wt % or more and 5 wt % or less of ethylbenzene.
   [3] The method for producing cumene according to [2] above, wherein the cumene purification step (f) includes a step of separating the solution (1) of the flow (1) into the solution (2) containing purified cumene, the solution (3) containing 2,3-dimethyl-2,3-diphenylbutane, and a solution (3′) containing ethylbenzene, and a step of mixing at least a part of the solution (3) with at least a part of the solution (3′) to obtain a mixed solution containing 2,3-dimethyl-2,3-diphenylbutane and ethylbenzene, the mixed solution being taken as the flow (3).
   [4] An apparatus for producing cumene using the method for producing cumene according to any one of [1] to [3] above, the apparatus including a facility A that separates the solution (1) of the flow (1) into at least a solution (2) containing purified cumene and a solution (3) containing 2,3-dimethyl-2,3-diphenylbutane.
   [5] The apparatus for producing cumene according to [4] above, wherein the facility A separates the solution (1) of the flow (1) further into a solution (3′) containing ethylbenzene.
   [6] The apparatus for producing cumene according to [4] or [5], wherein the facility A includes one distillation column.
   [7] The apparatus for producing cumene according to [4] or [5], wherein the facility A includes a plurality of distillation columns.
   [8] The apparatus for producing cumene according to any one of [5] to [7], further including a facility D that recovers cumene from the solution (3) and/or the solution (3′).
   [9] The apparatus for producing cumene according to [8] above, wherein the facility D includes one distillation column.
   [10] The apparatus for producing cumene according to [8] above, wherein the facility D includes a plurality of distillation columns.
   [11] A method for producing propylene oxide including the method for producing cumene according to any one of [1] to [3] above, the method for producing propylene oxide including steps (a) to (f) below,
an oxidation step (a): a step of oxidizing cumene to obtain cumene hydroperoxide,
an epoxidation step (b): a step of reacting propylene with cumene hydroperoxide obtained in the oxidation step (a) to obtain a reaction mixture containing propylene oxide and cumyl alcohol,
a separation step (c): a step of separating a mixture containing propylene oxide from the reaction mixture containing propylene oxide and cumyl alcohol obtained in the epoxidation step (b) to obtain a residue containing cumyl alcohol,
a propylene oxide purification step (d): a step of distilling the mixture containing propylene oxide separated by the separation step (c) to obtain purified propylene oxide,
a cumene conversion step (e): a step of converting cumyl alcohol in the residue containing cumyl alcohol obtained in the separation step (c) to cumene to obtain a solution (1) containing cumene, the solution (1) being taken as a flow (1), and
a cumene purification step (f): a step of separating the solution (1) of the flow (1) into at least a solution (2) containing purified cumene, and a solution (3) containing 2,3-dimethyl-2,3-diphenylbutane to obtain a flow (2) containing purified cumene and a flow (3) containing 2,3-dimethyl-2,3-diphenylbutane.

### EXAMPLE

Although the present invention will be hereinafter further specifically described with reference to Examples and Comparative Examples, the present invention is not limited to the following examples.

### Example 1

When 2,3-dimethyl-2,3-diphenylbutane, cumyl alcohol, and acetophenone were mixed in the formulation shown in Table 1 and maintained at 50° C., a solution with good flowability was obtained. When this solution was cooled to 21.4° C., good flowability was maintained.

### Example 2

When 2,3-dimethyl-2,3-diphenylbutane, and acetophenone were mixed in the formulation shown in Table 1 and maintained at a temperature of 50° C., a solution with good flowability was obtained. When this solution was cooled to 21.4° C., good flowability was maintained.

### Example 3

When 2,3-dimethyl-2,3-diphenylbutane, and acetophenone were mixed in the formulation shown in Table 1 and maintained at a temperature of 50° C., a solution with good flowability was obtained. When this solution was cooled to 21.4° C., good flowability was maintained.

### Comparative Example 1

When 2,3-dimethyl-2,3-diphenylbutane, cumyl alcohol, acetophenone, and naphtha cracked heavy oil were mixed in the formulation shown in Table 1 and maintained at a temperature of 50° C., a solution with good flowability was obtained. However, when the solution was cooled to 21.4° C., the solution solidified and lost flowability.

### Comparative Example 2

When 2,3-dimethyl-2,3-diphenylbutane, cumyl alcohol, and acetophenone were mixed in the formulation shown in Table 1, and maintained at a temperature of 50° C., a solution with good flowability was obtained. However, when this solution was cooled to 21.4° C., the solution solidified and lost flowability.

### Comparative Example 3

When 2,3-dimethyl-2,3-diphenylbutane, cumyl alcohol, and acetophenone were mixed in the formulation shown in Table 1, and maintained at a temperature of 50° C., a solution with good flowability was obtained. However, when this solution was cooled to 21.4° C., the solution solidified and lost flowability.

### Comparative Example 4

When 2,3-dimethyl-2,3-diphenylbutane, cumyl alcohol, and acetophenone were mixed in the formulation shown in Table 1, and maintained at a temperature of 50° C., a solution with good flowability was obtained. However, when this solution was cooled to 21.4° C., the solution solidified and lost flowability.

### Comparative Example 5

When 2,3-dimethyl-2,3-diphenylbutane, cumyl alcohol, and acetophenone were mixed in the formulation shown in Table 1, and maintained at a temperature of 50° C., a solution with good flowability was obtained. However, when this solution was cooled to 21.4° C., the solution solidified and lost flowability.

TABLE 1

Formulation (Wt %)

| | 2,3-dimethyl-2,3- | Cumyl | | |
|---|---|---|---|---|
| | diphenylbutane | alcohol | Acetophenone | Oil |
| Ex. 1 | 10 | 12 | 78 | 0 |
| Ex. 2 | 10 | 0 | 90 | 0 |
| Ex. 3 | 2 | 0 | 98 | 0 |
| C. Ex. 1 | 30 | 12 | 8 | 50 |
| C. Ex. 2 | 30 | 12 | 58 | 0 |
| C. Ex. 3 | 25 | 12 | 63 | 0 |
| C. Ex. 4 | 20 | 12 | 68 | 0 |
| C. Ex. 5 | 15 | 12 | 73 | 0 |

From the above results, it can be said that in the method for producing cumene that satisfies all the requirements of the present invention, the flow containing 2,3-dimethyl-2,3-diphenylbutane is capable of maintaining good flowability even at normal temperature.

1. A method for producing cumene comprising steps (e) and (f) below,
a cumene conversion step (e): a step of converting cumyl alcohol to cumene to obtain a solution (1) containing cumene, the solution (1) being taken as a flow (1), and
a cumene purification step (f): a step of separating the solution (1) of the flow (1) into at least a solution (2) containing purified cumene, and a solution (3) containing 2,3-dimethyl-2,3-diphenylbutane to obtain a flow (2) containing purified cumene and a flow (3) containing 2,3-dimethyl-2,3-diphenylbutane, wherein
the flow (3) contains 20 wt % or more and 99 wt % or less of acetophenone, and 1 wt % or more and 10 wt % or less of 2,3-dimethyl-2,3-diphenylbutane.

2. The method for producing cumene according to claim 1, wherein the flow (3) further contains 0.1 wt % or more and 5 wt % or less of ethylbenzene.

3. The method for producing cumene according to claim 2, wherein
the cumene purification step (f) includes a step of separating the solution (1) of the flow (1) into the solution (2) containing purified cumene, the solution (3) containing 2,3-dimethyl-2,3-diphenylbutane, and a solution (3′) containing ethylbenzene, and a step of mixing at least a part of the solution (3) with at least a part of the solution (3′) to obtain a mixed solution containing 2,3-dimethyl-2,3-diphenylbutane and ethylbenzene, the mixed solution being taken as the flow (3).

4. An apparatus for producing cumene using the method for producing cumene according to claim 1, the apparatus comprising a facility A that separates the solution (1) of the flow (1) into at least a solution (2) containing purified cumene and a solution (3) containing 2,3-dimethyl-2,3-diphenylbutane.

5. The apparatus for producing cumene according to claim 4, wherein the facility A separates the solution (1) of the flow (1) further into a solution (3′) containing ethylbenzene.

6. The apparatus for producing cumene according to claim 4, wherein the facility A comprises one distillation column.

7. The apparatus for producing cumene according to claim 4, wherein the facility A comprises a plurality of distillation columns.

8. The apparatus for producing cumene according to claim 5, further comprising a facility D that recovers cumene from the solution (3) and/or the solution (3′).

9. The apparatus for producing cumene according to claim 8, wherein the facility D comprises one distillation column.

10. The apparatus for producing cumene according to claim 8, wherein the facility D comprises a plurality of distillation columns.

11. A method for producing propylene oxide comprising the method for producing cumene according to claim 1, the method for producing propylene oxide comprising steps (a) to (f) below,
an oxidation step (a): a step of oxidizing cumene to obtain cumene hydroperoxide,
an epoxidation step (b): a step of reacting propylene with cumene hydroperoxide obtained in the oxidation step (a) to obtain a reaction mixture containing propylene oxide and cumyl alcohol,
a separation step (c): a step of separating a mixture containing propylene oxide from the reaction mixture containing propylene oxide and cumyl alcohol obtained in the epoxidation step (b) to obtain a residue containing cumyl alcohol,
a propylene oxide purification step (d): a step of distilling the mixture containing propylene oxide separated by the separation step (c) to obtain purified propylene oxide,
a cumene conversion step (e): a step of converting cumyl alcohol in the residue containing cumyl alcohol obtained in the separation step (c) to cumene to obtain a solution (1) containing cumene, the solution (1) being taken as a flow (1), and
a cumene purification step (f): a step of separating the solution (1) of the flow (1) into at least a solution (2) containing purified cumene, and a solution (3) containing 2,3-dimethyl-2,3-diphenylbutane to obtain a flow (2) containing purified cumene and a flow (3) containing 2,3-dimethyl-2,3-diphenylbutane.

12. The apparatus for producing cumene according to claim 5, wherein the facility A comprises one distillation column.

13. The apparatus for producing cumene according to claim 5, wherein the facility A comprises a plurality of distillation columns.
